(19) United States
(12) Patent Application Publication
(10) Pub. No.: US 2026/0216184 A1
(43) Pub. Date: Jul. 30, 2026

(54) USE OF COMPOUND 1 IN TREATMENT OR PREVENTION OF RENAL FIBROSIS AND OTHER DISEASES

(71) Applicant: SHENZHEN NEWDEL BIOTECH CO., LTD., Shenzhen, Guangdong (CN)

(72) Inventors: Xun HE, Shenzhen, Shenzhen, Guangdong (CN); Feng JIN, Shenzhen, Guangdong (CN); Yuhan GUO, Shenzhen, Guangdong (CN); Tianyuan ZHANG, Shenzhen, Guangdong (CN); Zhenwei LI, Shenzhen, Guangdong (CN); Wei HU, Shenzhen, Guangdong (CN)

(73) Assignee: SHENZHEN NEWDEL BIOTECH CO., LTD., Shenzhen, Guangdong (CN)

(21) Appl. No.: 19/147,302

(22) PCT Filed: Jan. 12, 2024
(86) PCT No.: PCT/CN2024/071954

§ 371 Date: Jul. 10, 2025

(30) Foreign Application Priority Data
Jan. 13, 2023 (CN) 202310077067.4

(51) Int. Cl. A61K 31/5025; A61P 13/12

(52) U.S. Cl. CPC A61K 31/5025; A61P 13/12

(57) ABSTRACT

The present invention relates to use of N-(3-chloro-5-(trifluoromethyl)phenyl)-3-((6-(4-hydroxypiperidin-1-yl)imidazo[1,2-b]pyridazin-3-yl)ethynyl)-2-methylbenzamide (also referred to herein as compound 1) or a pharmaceutically acceptable salt thereof in the treatment or prevention of renal fibrosis and other diseases.

### TECHNICAL FIELD

The present invention relates to use of N-(3-chloro-5-(trifluoromethyl)phenyl)-3-((6-(4-hydroxypiperidin-1-yl)imidazo[1,2-b]pyridazin-3-yl)ethynyl)-2-methylbenzamide (also referred to herein as compound 1) or a pharmaceutically acceptable salt thereof in the treatment or prevention of renal fibrosis and other diseases.

### BACKGROUND

Renal fibrosis is a common pathway and main pathological basis for the progression of various chronic kidney diseases (CKDs) to end-stage renal diseases (ESRDs), and is mainly manifested by glomerulosclerosis and renal interstitial fibrosis, including renal tubular cell injury, inflammatory cell infiltration, myofibroblast activation, tubular atrophy, and microvascular rarefaction. Firstly, fibroblasts and perivascular cells acquire myofibroblast phenotypes by expressing smooth muscle actin (α-SMA) and become the major matrix-producing cells. The activation of fibroblasts is accompanied by the infiltration of inflammatory cells, such as neutrophils, lymphocytes, and macrophages. Tubular epithelial cells change accordingly, resulting in tubular atrophy; capillary vascular endothelial cells undergo aging and apoptosis, resulting in microvascular rarefaction. Tubulointerstitial fibrosis is the main pathological basis for various kidney diseases and is also one of the important characterizations of chronic kidney diseases. The degree of fibrosis is closely related to the decline of renal function, thereby gradually leading to complete renal dysfunction. Patients may be asymptomatic in the early stages and are often diagnosed with renal fibrosis in the advanced stages, and the only treatment method at this time is dialysis or kidney transplantation. Delaying and preventing the formation of renal fibrosis is critical in the prevention and treatment of CKD. Until now, there is no substantial clinical practice to effectively treat chronic kidney diseases and other pathological changes. There are still no approved and marketed drugs for renal fibrosis, and there is a huge unmet clinical demand.

Two marketed drugs of the TRK inhibitor (larotrectinib and entrectinib) are both targeted at tumor patients with NTRK gene mutations. There are no marketed drugs for renal fibrosis indications, nor clinical trials and preclinical studies for renal fibrosis indications. There are no marketed drugs of the EPHA6 inhibitor, nor any clinical trial and preclinical study. For DDR1, there are only preclinical studies on idiopathic pulmonary fibrosis, but there are no drugs that enter the clinical trials for pulmonary fibrosis. Until now, there are no drugs targeting CSF1R go into clinical trials for fibrosis treatment.

The unilateral ureteral obstruction (UUO)-induced renal fibrosis model in mice is currently the most widely used renal fibrosis model. Tubulointerstitial fibrosis in C57BL/6J mice is induced by unilateral ureteral obstruction (UUO). This model can cause the obstruction of the renal drainage system in mice, lead to the acute renal function change and the chronic renal structure damage, and simulate the renal interstitial injury caused by the ureteral obstruction that is clinically common. Sustained increases of urinary tract pressure, renal blood flow reduction, venous drainage obstruction, macrophage infiltration, fibroblast proliferation, and scars formation result in tubulointerstitial fibrosis, tubular atrophy, and renal failure. This model is used in the present invention to test the therapeutic efficacy of the compound on renal fibrosis.

CN113831344B discloses N-(3-chloro-5-(trifluoromethyl)phenyl)-3-((6-(4-hydroxypiperidin-1-yl)imidazo[1,2-b]pyridazin-3-yl)ethynyl)-2-methylbenzamide.

### SUMMARY

The present invention relates to use of N-(3-chloro-5-(trifluoromethyl)phenyl)-3-((6-(4-hydroxypiperidin-1-yl)imidazo[1,2-b]pyridazin-3-yl)ethynyl)-2-methylbenzamide (also referred to herein as compound 1) or a pharmaceutically acceptable salt thereof in the treatment of renal fibrosis. In the present invention, renal fibrosis includes tubular and tubulointerstitial fibrosis as well as focal segmental and global glomerulosclerosis.

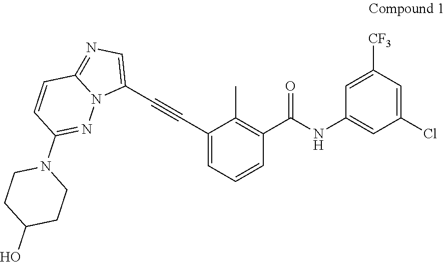 [SMILES: Cc1c(C#Cc2cnc3ccc(N4CCC(O)CC4)nn23)cccc1C(=O)Nc1cc(Cl)cc(C(F)(F)F)c1]

In a first aspect, the present invention provides use of compound 1 or a pharmaceutically acceptable salt thereof in the manufacture of a medicament for treating or preventing renal fibrosis.

In a second aspect, the present invention provides a method for treating or preventing renal fibrosis, comprising administering to a subject in need thereof a therapeutically effective amount of compound 1 or the pharmaceutically acceptable salt thereof.

In a third aspect, the present invention provides a pharmaceutical composition for treating or preventing renal fibrosis, comprising compound 1 or the pharmaceutically acceptable salt thereof, and a pharmaceutically acceptable excipient.

In a fourth aspect, the present invention provides compound 1 or a pharmaceutically acceptable salt thereof for use in the treatment or prevention of renal fibrosis.

The present invention also relates to use of compound 1 or the pharmaceutically acceptable salt thereof in the treatment or prevention of a disease mediated by DDR1, DDR2, CSF1R, and/or EPHA6 or that may be controlled by inhibiting DDR1, DDR2, CSF1R, and/or EPHA6.

In one aspect, the present invention provides use of compound 1 or the pharmaceutically acceptable salt thereof in the manufacture of a medicament for treating or preventing a disease mediated by DDR1 or that may be controlled by inhibiting DDR1.

In another aspect, the present invention provides a method for treating or preventing a disease mediated by DDR1 or that may be controlled by inhibiting DDR1, comprising administering to a subject in need thereof a therapeutically effective amount of compound 1 or the pharmaceutically acceptable salt thereof.

In another aspect, the present invention provides a pharmaceutical composition for treating or preventing a disease mediated by DDR1 or that may be controlled by inhibiting DDR1, comprising compound 1 or the pharmaceutically acceptable salt thereof, and a pharmaceutically acceptable excipient.

In another aspect, the present invention provides compound 1 or a pharmaceutically acceptable salt thereof for use in the treatment or prevention of a disease mediated by DDR1 or that may be controlled by inhibiting DDR1.

In each of the aspects described above, the disease mediated by DDR1 or that may be controlled by inhibiting DDR1 is fibrosis, tumors, immune disorders, skin and connective tissue diseases, musculoskeletal system diseases, genitourinary diseases, pregnancy complications, cardiovascular diseases, and neurodegenerative diseases.

In one aspect, the present invention provides use of compound 1 or the pharmaceutically acceptable salt thereof in the manufacture of a medicament for treating or preventing a disease mediated by DDR2 or that may be controlled by inhibiting DDR2.

In another aspect, the present invention provides a method for treating or preventing a disease mediated by DDR2 or that may be controlled by inhibiting DDR2, comprising administering to a subject in need thereof a therapeutically effective amount of compound 1 or the pharmaceutically acceptable salt thereof.

In another aspect, the present invention provides a pharmaceutical composition for treating or preventing a disease mediated by DDR2 or that may be controlled by inhibiting DDR2, comprising compound 1 or the pharmaceutically acceptable salt thereof, and a pharmaceutically acceptable excipient.

In another aspect, the present invention provides compound 1 or a pharmaceutically acceptable salt thereof for use in the treatment or prevention of a disease mediated by DDR2 or that may be controlled by inhibiting DDR2.

In each of the aspects described above, the disease mediated by DDR2 or that may be controlled by inhibiting DDR2 is fibrosis, tumors, immune system disorders, inflammatory diseases, musculoskeletal system diseases, skin and connective tissue diseases, digestive system diseases, cardiovascular diseases, genitourinary diseases, pregnancy complications, and neurodegenerative diseases.

In one aspect, the present invention provides use of compound 1 or the pharmaceutically acceptable salt thereof in the manufacture of a medicament for treating or preventing a disease mediated by CSF1R or that may be controlled by inhibiting CSF1R.

In another aspect, the present invention provides a method for treating or preventing a disease mediated by CSF1R or that may be controlled by inhibiting CSF1R, comprising administering to a subject in need thereof a therapeutically effective amount of compound 1 or the pharmaceutically acceptable salt thereof.

In another aspect, the present invention provides a pharmaceutical composition for treating or preventing a disease mediated by CSF1R or that may be controlled by inhibiting CSF1R, comprising compound 1 or the pharmaceutically acceptable salt thereof, and a pharmaceutically acceptable excipient.

In another aspect, the present invention provides compound 1 or a pharmaceutically acceptable salt thereof for use in the treatment or prevention of a disease mediated by CSF1R or that may be controlled by inhibiting CSF1R.

In each of the aspects described above, the disease mediated by CSF1R or that may be controlled by inhibiting CSF1R is fibrosis, tumors, immune system disorders, inflammatory diseases, and neurodegenerative diseases.

In one aspect, the present invention provides use of compound 1 or the pharmaceutically acceptable salt thereof in the manufacture of a medicament for treating or preventing a disease mediated by EPHA6 or that may be controlled by inhibiting EPHA6.

In another aspect, the present invention provides a method for treating or preventing a disease mediated by EPHA6 or that may be controlled by inhibiting EPHA6, comprising administering to a subject in need thereof a therapeutically effective amount of compound 1 or the pharmaceutically acceptable salt thereof.

In another aspect, the present invention provides a pharmaceutical composition for treating or preventing a disease mediated by EPHA6 or that may be controlled by inhibiting EPHA6, comprising compound 1 or the pharmaceutically acceptable salt thereof, and a pharmaceutically acceptable excipient.

In another aspect, the present invention provides compound 1 or a pharmaceutically acceptable salt thereof for use in the treatment or prevention of a disease mediated by EPHA6 or that may be controlled by inhibiting EPHA6.

In each of the aspects described above, the disease mediated by EPHA6 or that may be controlled by inhibiting EPHA6 is kidney diseases, tumors, digestive system diseases, cardiovascular diseases, and psychiatric diseases.

### BRIEF DESCRIPTION OF THE DRAWINGS

FIG. 1 shows changes in the body weight of mice.

FIG. 2 shows H&E staining results of kidney tissues.

FIG. 3A shows Masson staining results of collagen fibers in kidney tissues, and FIG. 3B shows quantitative statistical analysis results of Masson staining.

FIG. 4A shows α-SMA immunohistochemical staining results of kidney tissues, and FIG. 4B shows quantitative statistical analysis results of α-SMA immunohistochemical staining.

FIG. 5A shows immunohistochemical staining results of F4/80 in kidney tissues, and FIG. 5B shows quantitative statistical analysis results of immunohistochemical staining of F4/80.

### DETAILED DESCRIPTION

The present invention is further illustrated by, but not limited to, the following examples that explain the present invention.

### Example 1: Inhibitory Effect of Compound 1 on Kinase Activities of TRK, DDR, EPHA6, and CSF1R

### Assay Principle of HTRF Method:

A kinase phosphorylates a substrate, and an antibody labeled with Eu-CryPtate binds to a phosphorylation site in the substrate; streptavidin-XL665 binds to the substrate biotin; when Eu and XL665 are close, Eu as a donor emits emission light (620 nm) after being excited by a light source (320 nm) and transfers energy resonance to a close XL665 receptor, and the receptor emits emission light (665 nm) after being excited; the specific signal is proportional to the phosphorylated substrate; when an inhibitor is added, the phosphorylation level is inhibited, the emission light at 665 nm is undetectable, and only the emission light at 620 nm is detected, so as to evaluate the level of inhibition of kinase activity by the compound.

### TRKA Enzyme Activity Assay:

Compound 1 was serially diluted in DMSO from a starting concentration of 1 μM to obtain a total of 10 concentrations after four-fold dilution. Replicate wells were set for the assay. 25 nL of the compound was transferred to a 384 reaction plate (Catalog No. 784075, Greiner) using Echo 665. 2× kinase solution was prepared with 1× kinase reaction buffer (5× buffer, 5 mM MgCl₂, 1 mM DTT, H₂O, and 1 mM MnCl₂). 2.5 μL of TRKA (1.5 nM, 08-186, Carna) solution was transferred to the 384 reaction plate. The plate was centrifuged at 1000 rpm for 60 s using a centrifuge and incubated at 25° C. for 10 min. A mixed solution of 2× biotin-labeled tyrosine kinase substrate (1 M) (Catalog No. 61TKOBLE, Cisbio—PerkinElmer) and ATP (25 M) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### TRKB Enzyme Activity Assay:

Compound 1 was serially diluted in DMSO from a starting concentration of 1 μM to obtain a total of 10 concentrations after four-fold dilution. Replicate wells were set for the assay. 25 nL of the compound was transferred to a 384 reaction plate using Echo 665. 2× kinase solution was prepared with 1× kinase reaction buffer (5× buffer, 5 mM MgCl₂, 1 mM DTT, H₂O, and 1 mM MnCl₂). 2.5 μL of TRKB (1.4 nM, 08-187, Carna) solution was transferred to the 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### TRKC Enzyme Activity Assay:

Compound 1 was serially diluted in DMSO from a starting concentration of 1 μM to obtain a total of 10 concentrations after four-fold dilution. Replicate wells were set for the assay. 2× kinase solution was prepared with 1× kinase reaction buffer (5× buffer, 5 mM MgCl₂, 1 mM DTT, H₂O, and 1 mM MnCl₂). 2.5 μL of TRKC (1.5 nM, 08-197, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### DDR1 Enzyme Activity Assay:

Compound 1 was serially diluted in DMSO from a starting concentration of 1 μM to obtain a total of 10 concentrations after four-fold dilution. Replicate wells were set for the assay. 2× kinase solution was prepared with 1× kinase reaction buffer (5× buffer, 5 mM MgCl₂, 1 mM DTT, H₂O, and 1 mM MnCl₂). 2.5 μL of DDR1 (2.7 nM, 08-113, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### DDR2 Enzyme Activity Assay:

Compound 1 was serially diluted in DMSO from a starting concentration of 1 μM to obtain a total of 10 concentrations after four-fold dilution. Replicate wells were set for the assay. 2× kinase solution was prepared with 1× kinase reaction buffer (5× buffer, 5 mM MgCl₂, 1 mM DTT, H₂O, and 1 mM MnCl₂). 2.5 μL of DDR2 (1.3 nM, 08-114, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### EPHA6 Enzyme Activity Assay:

Compound 1 was serially diluted in DMSO from a starting concentration of 50 μM to obtain a total of 10 concentrations after four-fold dilution. Replicate wells were set for the assay. 2× kinase solution was prepared with 1× kinase reaction buffer (5× buffer, 5 mM MgCl₂, 1 mM DTT, H₂O, and 1 mM MnCl₂). 2.5 μL of EPHA6 (0.5 nM, 08-125, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### CSF1R Enzyme Activity Assay:

Compound 1 was serially diluted in DMSO from a starting concentration of 10 μM to obtain a total of 10 concentrations after four-fold dilution. Replicate wells were set for the assay. 2× kinase solution was prepared with 1× kinase reaction buffer (5× buffer, 5 mM MgCl₂, 1 mM DTT, H₂O, and 1 mM MnCl₂). 2.5 μL of CSF1R (1.3 nM, 08-114, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

The IC₅₀s of compound 1 against DDR1, DDR2, TRKA, TRKB, TRKC, EPHA6, and CSF1R obtained from the assays described above are listed in Table 1.

TABLE 1
IC₅₀s of compound 1 against DDR1, DDR2,
TRKA, TRKB, TRKC, EPHA6, and CSF1R

| | IC₅₀ (nM) |
|---|---|
| DDR1 | 0.7616 |
| DDR2 | 37.93 |
| TRKA | 4.397 |
| TRKB | 0.4325 |
| TRKC | 0.350 |
| EPHA6 | 12.84 |
| CSF1R | 14.71 |

### Example 2. Evaluation of Therapeutic Effect of Compound 1 on UUO-Induced Renal Fibrosis in Mice

### Method

The pharmacodynamic experiment was designed into 7 groups, which were groups receiving the following treatments:
Group 1: sham group;
Group 2: vehicle group;
Group 3: compound 1, 3 mg/kg QD (hereinafter, mg/kg was abbreviated as mpk);
Group 4: compound 1, 10 mg/kg QD;
Group 5: compound 1, 30 mg/kg QD;
Group 6: compound 1, 100 mg/kg QD; and
Group 7: positive drug pirfenidone, 250 mg/kg BID (hereinafter, pirfenidone was abbreviated as PFD).

Modeling a UUO model mouse: C57 mice (male, 8 weeks old, weighing 20-25 g) were purchased from Zhejiang Vital River Laboratory Animal Technology Co., Ltd. After anesthesia, a 1-2 cm incision was made in the left kidney region of the abdomen, and the intestinal tract was carefully removed to expose the ureter. The ureter was ligated with double lines and cut off at the middle position of the double-line ligation. The intestinal tract was recovered, and the incision was stitched.

Mice in the sham group: C57 mice were anesthetized, a 1-2 cm incision was made in the left kidney region of the abdomen, and the intestinal tract was carefully removed to expose the ureter. The ligation was not performed. The intestinal tract was recovered, and the incision was stitched.

All animals were administered orally on the day of surgery. Animals in the sham group and vehicle group were administered a test drug vehicle (5% DMSO+5% Tween80+20% PEG400+70% H₂O). All administration solutions were freshly prepared before use. The experiment ended 10 days after the administration.

TABLE 2
Pharmacodynamic grouping and administration regimens

| | | | | Administration | | |
|---|---|---|---|---|---|---|
| | Number | | Administration | volume | | |
| Group | of | Administration | dose | parameter | Route of | Frequency of |
| No. | animals | group | (mg/kg) | (μL/g) | administration | administration |
| 1 | 5 | Sham | — | 10 | P.O | QD |
| 2 | 10 | Vehicle | — | 10 | P.O | QD |
| 3 | 10 | Compound 1 | 3 | 10 | P.O | QD |
| 4 | 10 | Compound 1 | 10 | 10 | P.O | QD |
| 5 | 10 | Compound 1 | 30 | 10 | P.O | QD |
| 6 | 10 | Compound 1 | 100 | 10 | P.O | QD |
| 7 | 10 | Pirfenidone | 250 | 10 | P.O | BID |

The preparation method for the test compound included weighing a free base compound 1 powder, adding the powder to 5% DMSO, and performing vortex mixing until the mixture was clear; adding 5% Tween-80, and performing vortex mixing; adding 20% PEG400, and performing vortex mixing; and adding 70% pure water, and performing vortex mixing for later use. The preparation method for the positive drug included weighing pirfenidone into a mortar, adding a small amount of 0.5% CMC-Na, grinding the mixture fully and uniformly, supplementing the mixture with the remaining 0.5% CMC-Na, and mixing well for use.

At the end of the experiment, the left kidney was taken and placed in 4% PFA for fixation. The kidney was conventionally sectioned and stained 24 h after the fixation.

H&E staining: the sections were conventionally dewaxed and rehydrated, stained with hematoxylin for 20 min, differentiated with 1% hydrochloric acid in ethanol, and returned to blue with running water for 20 min; the sections were treated with 70% ethanol, 80% ethanol, and 90% ethanol for 3 min, respectively, and stained with eosin for 10 s; and the sections were then treated with 95% ethanol and 100% ethanol for 10 min, respectively, subjected to permeabilization with xylene, mounted with neutral balsam, and observed under a common optical microscope.

Masson staining: the sections were conventionally dewaxed and rehydrated, stained with a nuclear staining solution for 1 min, and washed with a washing solution for 30 s; the sections were stained with a cytoplasmic staining solution for 10 s, and washed with a washing solution for 30 s; the sections were differentiated with a differentiation solution for 8 min; after the differentiation solution was discarded, the sections were directly stained with a counterstain solution for 3 min, washed with 100% ethanol to remove the unfixed stains, subjected to permeabilization with xylene, mounted with neutral balsam, and observed under a common optical microscope.

α-SMA immunohistochemical staining: paraffin sections of kidney tissues were dewaxed and rehydrated; a Tris-EDTA antigen repair solution was added and heated for antigen repair; endogenous hydroperoxides were removed; the sections were blocked at room temperature for 1 h, incubated with primary antibody α-SMA (1:500) at room temperature for 1 h, and then incubated with a secondary antibody at room temperature for 1 h; the sections were subjected to color development with DAB for 3-5 min, stained with hematoxylin for 3 min, differentiated in a differentiation solution for 30 s, returned to blue in a blue returning solution, dehydrated for permeabilization, and mounted with neutral balsam.

F4/80 immunohistochemical staining: paraffin sections of kidney tissues were dewaxed and rehydrated; a Tris-EDTA antigen repair solution was added and heated for antigen repair; endogenous hydroperoxides were removed; the sections were blocked at room temperature for 1 h, incubated with primary antibody F4/80 (1:1000) at room temperature for 1 h, and then incubated with a secondary antibody at room temperature for 1 h; the sections were subjected to color development with DAB for 3-5 min, stained with hematoxylin for 3 min, differentiated in a differentiation solution for 30 s, returned to blue in a blue returning solution, dehydrated for permeabilization, and mounted with neutral balsam. [0075]5-9 fields were collected at random for each sample, and ImageJ was used to perform area analysis of Masson positive areas. The average value calculated from the fields collected for each sample was the final statistical data for this sample. The results were statistically analyzed using GraphPad_Prism 8 software, and data were analyzed by one-way analysis of variance (One way ANOVA). p<0.05 was defined as a significant difference.

### Result

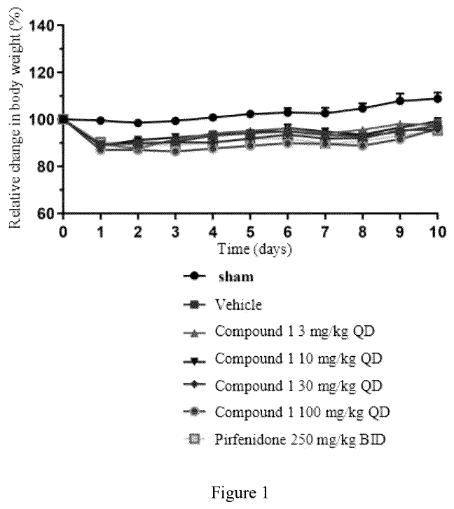

The relative changes in the body weight of mice in each group fluctuated within the normal range are shown in FIG. 1 and Table 3.

TABLE 3
Relative body weight changes of mice

| Time | Group | | | | | | |
|---|---|---|---|---|---|---|---|
| (days) | Group 1 | Group 2 | Group 3 | Group 4 | Group 5 | Group 6 | Group 7 |
| 0 | 100 ± 0.00% | 100 ± 0.00% | 100 ± 0.00% | 100 ± 0.00% | 100 ± 0.00% | 100 ± 0.00% | 100 ± 0.00% |
| 1 | 99.47 ± 0.71% | 89.36 ± 0.57% | 89.36 ± 0.77% | 88.83 ± 0.43% | 89.24 ± 0.48% | 86.92 ± 0.52% | 90.41 ± 0.25% |
| 2 | 98.40 ± 0.57% | 89.60 ± 1.33% | 87.58 ± 1.52% | 91.11 ± 1.12% | 89.95 ± 0.81% | 86.94 ± 1.15% | 89.78 ± 0.81% |
| 3 | 99.33 ± 0.94% | 90.49 ± 1.04% | 91.41 ± 1.00% | 92.22 ± 0.81% | 90.15 ± 0.71% | 86.29 ± 1.30% | 88.90 ± 0.75% |
| 4 | 100.73 ± 1.38% | 92.96 ± 082% | 93.99 ± 0.64% | 93.43 ± 0.63% | 90.11 ± 047% | 87.52 ± 1.24% | 90.19 ± 0.85% |
| 5 | 102.23 ± 1.61% | 94.05 ± 0.83% | 95.11 ± 0.69% | 93.97 ± 0.75% | 91.83 ± 0.53% | 88.74 ± 1.62% | 91.53 ± 0.62% |
| 6 | 102.80 ± 1.99% | 94.82 ± 0.46% | 96.13 ± 1.83% | 96.35 ± 1.01% | 93.43 ± 0.40% | 89.77 ± 1.77% | 91.83 ± 0.85% |
| 7 | 102.53 ± 2.43% | 93.34 ± 0.53% | 93.87 ± 0.86% | 94.67 ± 1.82% | 91.79 ± 0.50% | 89.47 ± 1.59% | 89.72 ± 0.68% |
| 8 | 104.63 ± 2.12% | 92.79 ± 1.06% | 95.49 ± 0.60% | 93.35 ± 1.56% | 91.98 ± 0.55% | 88.75 ± 1.77% | 90.68 ± 0.85% |
| 9 | 107.81 ± 3.14% | 94.72 ± 0.81% | 98.10 ± 0.97% | 96.52 ± 1.74% | 95.25 ± 0.68% | 91.50 ± 2.51% | 93.04 ± 0.66% |
| 10 | 108.74 ± 2.65% | 97.32 ± 1.07% | 97.57 ± 2.16% | 99.08 ± 1.36% | 95.69 ± 0.44% | 95.83 ± 2.60% | 96.16 ± 1.10% |

Relative body weight change=body weight on day n/body weight on day 0×100%

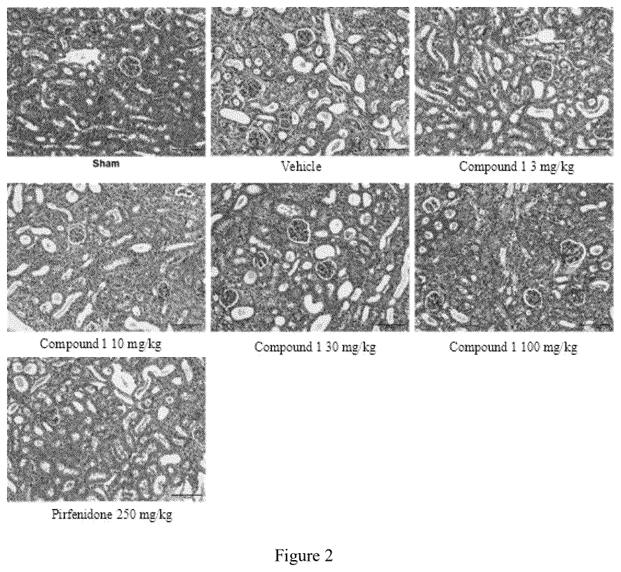

In the H&E staining, the mouse in the sham group had a full kidney and clear tubular and glomerular structures. In the vehicle group, the kidney had significant tubular atrophy, tubular flattening, tighter glomerulus, and thickened glomerular mesangium, and free colorable substances appeared in the renal tubule. Compared with the vehicle group, the compound 1 dose groups showed reduced tubular atrophy, a looser glomerular structure, and no significant thickening of tubular basement membrane. Compared with the vehicle group, the pirfenidone group showed a looser glomerular structure and no significant material deposition in the renal tubular spaces (FIG. 2).

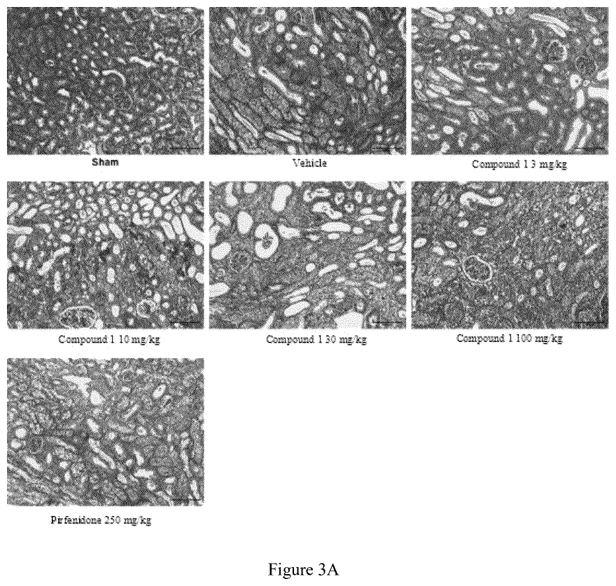

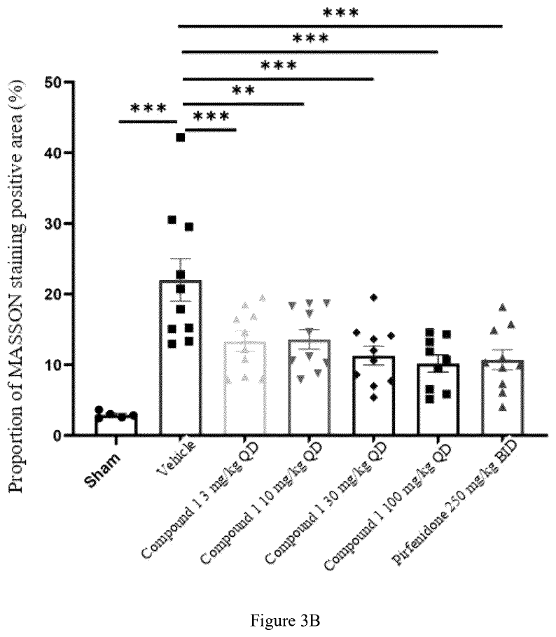

Masson collagen fiber staining results showed that in the sham group, the area of positive staining was very small, mainly in the lumen of the renal tubule, which was the brush border of the proximal tubule. In the vehicle group, there was a large amount of positive staining in the renal tubular spaces, which was the collagen deposition in the tubulointerstitium and a typical pathological characteristic of tubulointerstitial fibrosis. Compound 1 showed a significant effect in relieving the collagen deposition in the renal tubular spaces in the 3 mpk, 10 mpk, 30 mpk, and 100 mpk groups (FIG. 3A). The quantitative analysis results showed that compound 1 could significantly reduce the Masson staining positive area at the dose of 3 mpk, with a certain dose-dependent relationship exhibited at different doses (FIG. 3B). The compound at the dose of 30 mpk QD could achieve the similar efficacy to that of the positive drug pirfenidone at 250 mpk BID, and the administration dose and frequency had significant advantages.

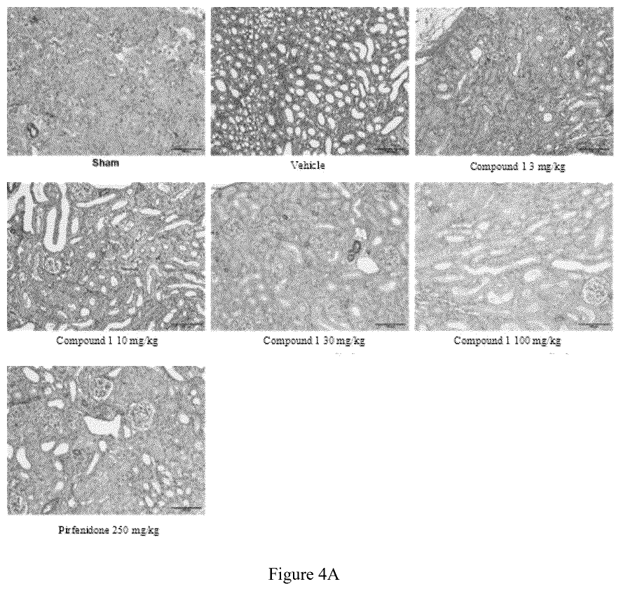

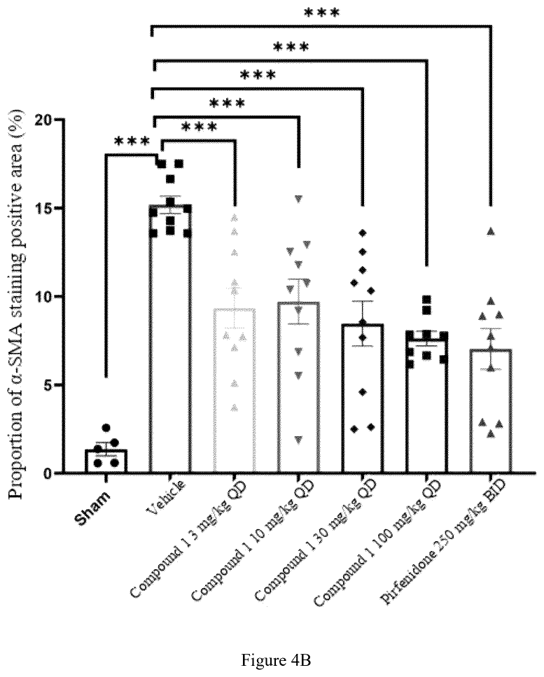

α-SMA (Alpha-smooth muscle actin) is one of the markers of extracellular matrixes during fibrosis and can directly reflect the level of fibrosis. The levels of α-SMA in the kidneys were evaluated after intervention in each group by an immunohistochemical method (FIG. 4A). The results showed that compared with the sham group, the histochemical area of α-SMA in the vehicle group was significantly increased (P<0.01), indicating a significant increase in fibers; compared with the vehicle group, the positive area of α-SMA was significantly reduced at 3 mpk (P<0.05), and the levels of α-SMA were significantly reduced at 10 mpk, 30 mpk, and 100 mpk in the compound administration groups, showing a certain dose dependence (FIG. 4B).

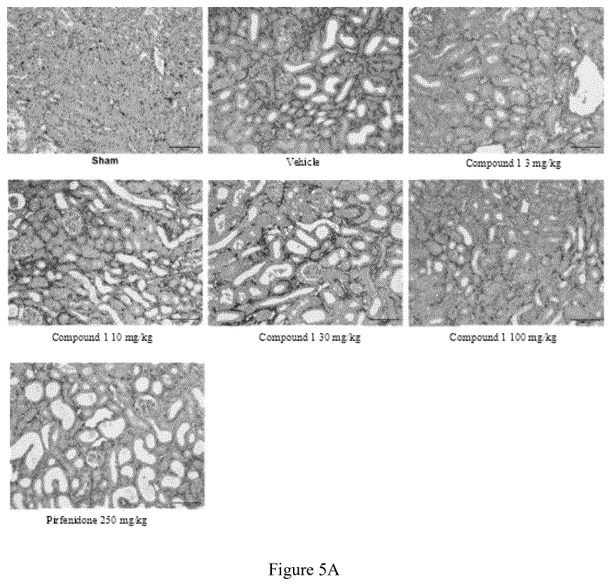

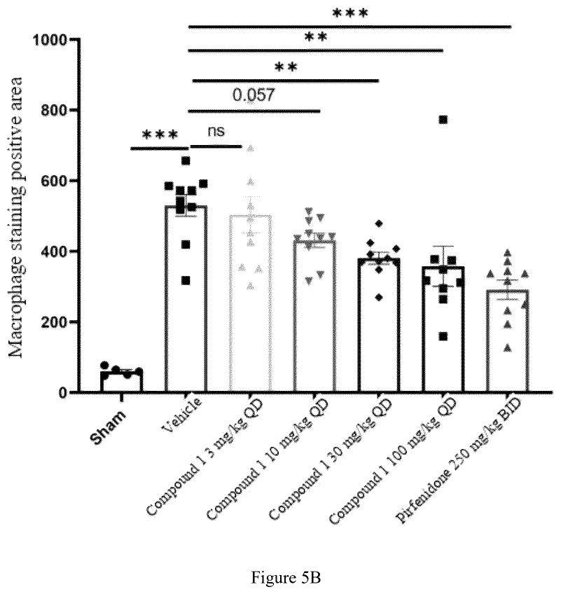

Macrophages, particularly M2 type macrophages, are considered to play an important role in the development and progression of tissue fibrosis, and can serve as a reference for inflammatory responses. F4/80 is a marker for mature macrophages. The expression of macrophages in the kidney tissues after different types of interventions was assayed by the immunohistochemical method. The results are shown in FIGS. 5A and 5B. In the sham group, macrophages in the kidney were rarely expressed and mainly accumulated in the tubulointerstitium, which were tissue-resident macrophages. In the UUO model group, i.e., the vehicle group, the expression levels of macrophages in the kidney were remarkably increased and accumulated more around the injured renal tubules. Compared with the vehicle group, each dose group of the compound showed reduced expression levels of macrophages and significantly reduced at 30 mpk and 100 mpk.

### CONCLUSION

In the experiment, the collagens, fibrosis factors and macrophages were significantly increased in the vehicle group, indicating that the building of the mouse renal fibrosis model is successful. Test compound 1 significantly reduced the production of collagens (Masson staining) and fibrosis factors (α-SMA) in the kidney, significantly inhibited the expression levels of macrophages (F4/80 staining), showing significant anti-fiboris and anti-inflammation effect in a dose-dependent manner. Test compound 1 showed a significant effect in relieving renal fibrosis at the dose of 3 mg/kg, which could be considered as an onset dose of the test compound 1 for treating renal fibrosis. Test compound 1 had significant advantages in dosage and administration compared with the positive drug pirfenidone.

Note: in FIGS. 3B, 4B, and 5B, ns indicates no significant difference, * indicates p<0.05, **indicates p<0.01, and *** indicates p<0.001.

The aforementioned examples and descriptions of certain embodiments should be construed as illustrating, rather than as limiting, the present invention as defined by the claims. As will be readily understood, many changes and combinations of the features described above can be employed without departing from the present invention as set forth in the claims. All such changes are intended to be included within the scope of the present invention. All cited references are incorporated herein by reference in their entirety.

What is claimed is:

1. Use of compound 1 or a pharmaceutically acceptable salt thereof in the manufacture of a medicament for treating or preventing renal fibrosis, wherein compound 1 has the following structure:

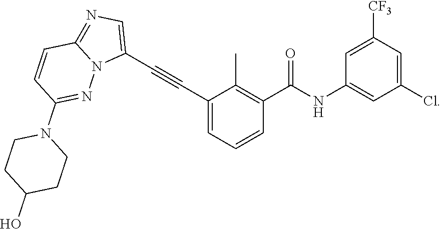 [SMILES: Cc1c(C#Cc2cnc3ccc(N4CCC(O)CC4)nn23)cccc1C(=O)Nc1cc(Cl)cc(C(F)(F)F)c1]

2. Use of compound 1 or a pharmaceutically acceptable salt thereof in the manufacture of a medicament for treating or preventing a disease mediated by DDR1 or that may be controlled by inhibiting DDR1, wherein compound 1 has the following structure:

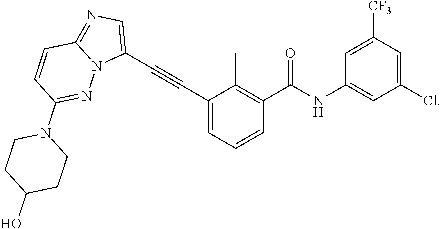 [SMILES: Cc1c(C#Cc2cnc3ccc(N4CCC(O)CC4)nn23)cccc1C(=O)Nc1cc(Cl)cc(C(F)(F)F)c1]

3. Use of compound 1 or a pharmaceutically acceptable salt thereof in the manufacture of a medicament for treating or preventing a disease mediated by DDR2 or that may be controlled by inhibiting DDR2, wherein compound 1 has the following structure:

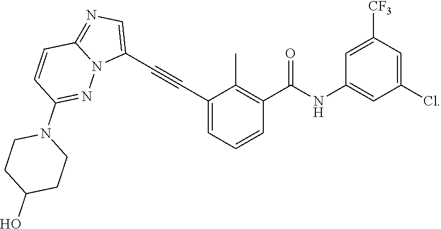 [SMILES: Cc1c(C#Cc2cnc3ccc(N4CCC(O)CC4)nn23)cccc1C(=O)Nc1cc(Cl)cc(C(F)(F)F)c1]

4. Use of compound 1 or a pharmaceutically acceptable salt thereof in the manufacture of a medicament for treating or preventing a disease mediated by EPHA6 or that may be controlled by inhibiting EPHA6, wherein compound 1 has the following structure:

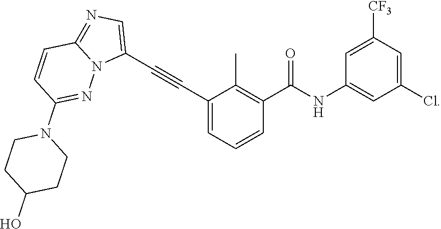 [SMILES: Cc1c(C#Cc2cnc3ccc(N4CCC(O)CC4)nn23)cccc1C(=O)Nc1cc(Cl)cc(C(F)(F)F)c1]

5. Use of compound 1 or a pharmaceutically acceptable salt thereof in the manufacture of a medicament for treating or preventing a disease mediated by CSF1R or that may be controlled by inhibiting CSF1R, wherein compound 1 has the following structure: 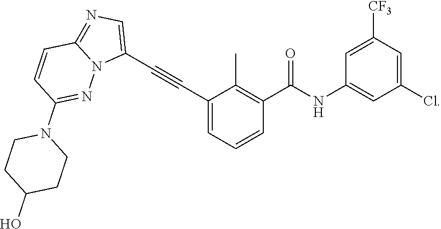 [SMILES: Cc1c(C#Cc2cnc3ccc(N4CCC(O)CC4)nn23)cccc1C(=O)Nc1cc(Cl)cc(C(F)(F)F)c1]
